(19) United States
(12) Patent Application Publication
(10) Pub. No.: US 2014/0046600 A1
(43) Pub. Date: Feb. 13, 2014

(54) SIM CARD BASED MEDICAL TESTING AND DATA TRANSMISSION SYSTEM

(71) Applicant: Netanel Avner, Mevaseret (IL)

(72) Inventor: Netanel Avner, Mevaseret (IL)

(21) Appl. No.: 13/961,031

(22) Filed: Aug. 7, 2013

Related U.S. Application Data
(60) Provisional application No. 61/680,316, filed on Aug. 7, 2012.

(51) Int. Cl. G01N 33/487

(52) U.S. Cl. CPC G01N 33/48792
USPC 702/19

(57) ABSTRACT

The present invention is a system for measuring and transmitting measuring a characteristic of a physiological fluid and transmitting said characteristic to a receiver, having a card constructed and arranged to perform analysis of a physiological fluid and said card configured to convert said analysis to an electronic file; a transmission device constructed and arranged to receive said card and transmit said electronic data file; and a receiver configured to be operatively associated with said transmission device for receiving said data transmitted thereto, wherein said receiver prepares an output based on reception of said electronic data.

### INDEX TO RELATED APPLICATIONS

This application is a non-provisional of and claims benefit to U.S. Provisional Patent Application Serial No. 61/680,316 filed Aug. 7, 2012 the disclosure of which is incorporated herein by reference in its entirety.

### BACKGROUND OF THE INVENTION

Medical testing is often an important aspect in the treatment of a patient. However, when medical testing involves repeated testing of physiological fluids patient compliance is typically a significant issue. This is because the patient is often required to physically present themselves at the laboratory or other testing facility in order to have a sample presented, have it processed and have the test results forwarded to an appropriate person. Patient compliance can be greatly improved if patients were not required to physically present themselves at a testing center. The present invention addresses the need for patent compliance by providing a system whereby a patient can obtain a sample of their own physiological fluid, have that sample tested in the location where they are currently found and have the test result electronically transmitted. In configuring a system in this manner, patient compliance is increased and medical/health care providers receive significantly increased data relating to testing of physiological fluids.

### SUMMARY OF THE INVENTION

In one embodiment, the present invention is a system whereby a disposable medical testing strip is incorporated onto a conventional SIM card. The SIM card platform will further electronically transmit a reading of a medical test. In one embodiment, the transmission is over a standard GSM mobile apparatus to a centralise server that analyse the data and relay it to the proper party, (Doctor, Lab, Medical provider, Police and the like).

According to the present invention, a dedicated SIM card will be provided that will include 2 functional components:
1. A standard SIM card with standard GSM related functionality and information (i.e. IMSI, KI,)
2. A medical testing system that includes a specimen reading panel capable of reading a specific specimen and detecting the relevant chemicals, converting the reading to an electronic data and transmitting over the GSM network.

The SIM card is disposable and specific to various chemicals elements (i.e. a different testing apparatus will be created to analyse a specific reading).

These medical analysis test SIM cards, can test for presence of various chemicals in a specimen (blood, Urine, saliva and other type of specimen) this analysis SIM card can detect Glucose, Protein pH, Leukocytes, Nitrites, Ketones, Bilirubin, Blood, Urobilinogen, and Specific Gravity and more type of chemicals are simple, easy to use sim card based strips for the detection of key diagnostic chemical markers in specimens. They will provide the the doctors, laboratories and healthcare professionals with preliminary diagnosis of, and initial screening for potential health problems. Remotely, Medical testing SIM card are plastic strips to which chemically specific

SIM card pads are affixed. The SIM card pads react with the sample specimen to provide a standardized electronic code that then is transmitted through the GSM sim card over the GSM network immediately to a server that analyse the data remotely and provide immedite reply to the handset as well as to the treating Doctor, lab and other interested parties over the Internet. With the accurate level of each chemical factor. Test results may provide useful information regarding carbohydrate (sugar) metabolism (diabetes), kidney function, acid-base balance, bacteraemia, occult blood, high leukocytes (infection) and other conditions of overall health. The SIM card strips are ready to use upon removal from the vial and the entire strip is disposable.

In one embodiment, the present invention includes a method of interfacing and integrating between a GSM SIM card and a medical analysis testing apparatus.

The integrated SIM card medical analisys apparatus will have the ability to analyze and transmit over the GSM network a variety of chemical readings and variety of specimens for medical or other response and uses.
a. A unique apparatus that enable users to place a tested specimen onto a medical apparatus and transmit the reading to a remote server/location over the GSM network, using a standard GSM mobile handset.
b. The medical testing apparatus

The system will transmit the reading to a remote location over the GSM network in USSD or GPRS data or SMS or voice method.

In one embodiment, the present invention is a method for testing biological fluids, formatting test results for transmission, transmitting test data, said method comprising the steps of: providing a SIM card according to claim 1;

placing a test fluid on a test region indicated on said SIM card;

covering said SIM card with a cover on top of said test fluid;

inserting said SIM card in a device configured for reading test results from said SIM card;

formatting said test results into data for transmission;

transmitting said test results data on a transmitting device for transmitting said data;

receiving said transmitted data on a receiving device configured for receiving said transmitted data;

storing said data on a storage device configured for storing said data.

The method test fluid is at least one of blood, saliva, urine or mucosal fluid.

The method further includes an output of said stored data. The output is electively configured and can be varied to any deisred parameters.

### BRIEF DESCRIPTION OF THE SEVERAL VIEWS OF THE DRAWING

FIG. 1 is a plan view of the medical testing side of the SIM card of the present invention.

FIG. 2 is a plan view of the transmission side of the SIM card of the present invention.

FIG. 3 is a plan view of the medical testing side of the SIM card of the present invention with a cover for covering the medical specimen partially positioned over the SIM card.

FIG. 4 is schematic of one embodiment of the present invention.

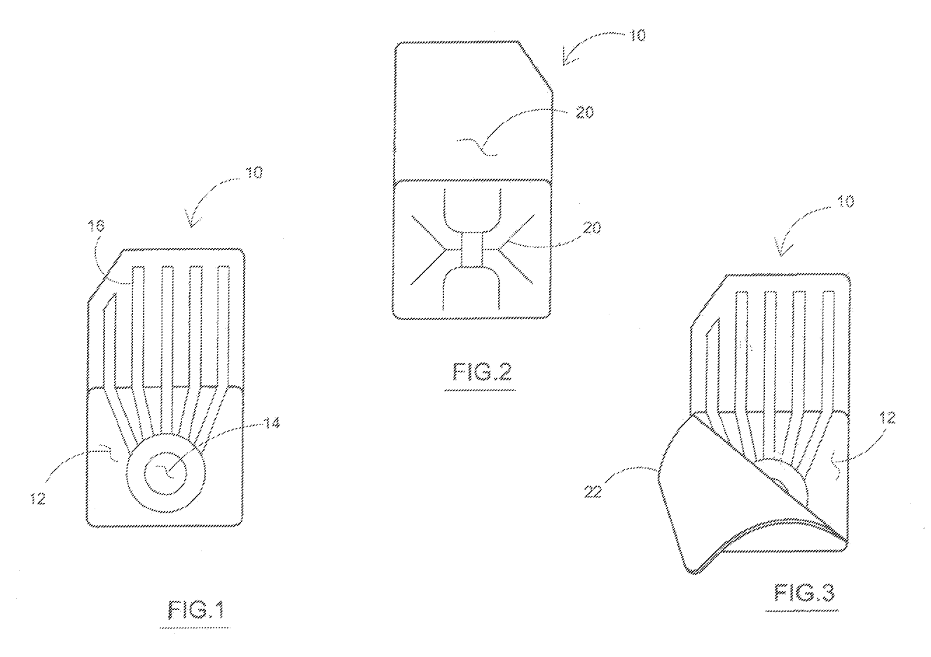

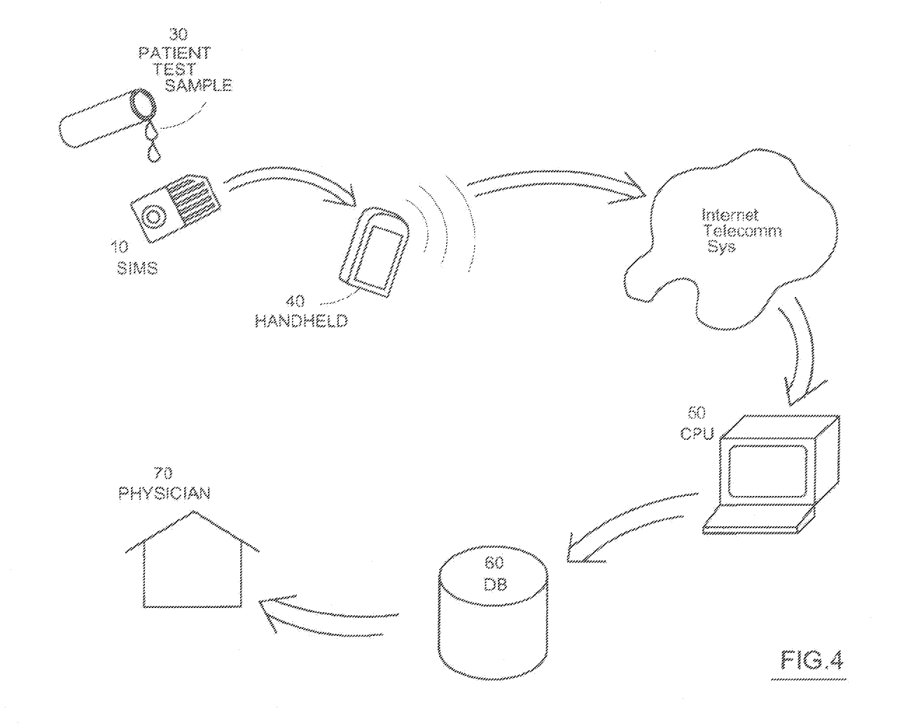

### DETAILED DESCRIPTION OF THE PREFERRED EMBODIMENT

The present invention provides a SIM card 10 constructed and arranged to be used in the system of the present invention for performing various medical tests. The SIM card 10 has a first side 12 with a region 14 constructed and arranged for the placement and testing of various biological chemicals. Testing strips and devices are known and the present invention contemplates use of known test methods including, but not limited to colorimetric, chemiluminescence, electrochemical, and others that are configured to provide test results and information to first circuitry 16. First side 12 further has first circuitry 16 for converting the test results into a electronic data whereby said data is configured for transmission. The SIM card 10 has a second side 18 with second circuitry 20. In one embodiment, second circuitry 20 is operatively associated with first circuitry 16 and is configured for processing data from said first circuitry 16. Said second circuitry 20 further is operatively associated with a transmission device to transmit said data to a receiver 50. Although FIG. 4 demonstrates receiver 50 as a computer processing unit, it is contemplated that any device configured to receive transmitted data is contemplated in the present invention.

In a preferred embodiment, the SIM card 10 is constructed and arranged to determine quantitative and/or qualitative chemical data from a biological sample placed thereon. Biological sample, as generally understood, refers to any biological/physiological fluid. The physiological fluid includes but would not be limited to, saliva, blood, urine and the like. It is further understood and contemplated in the present invention that universal precautions be utilized when handling physiological samples. As generally understood in the present invention, SIM card testing device 10 is provided to the user.

In one embodiment, the invention includes a separate testing strip incorporated with a SIM card. The testing strip is either attached to a SIM card or inserted with a SIM card. In this embodiment, the strip and SIM card are each configured to interact with one another and provide for testing results to be transferred to the SIM card for transmission. As one non-limiting example, a glucose testing strip is provided with test result transfer means for transferring test results to the SIM card. In one embodiment, the testing strip includes incorporated electronic circuitry to interact with circuitry 16 on SIM card 10.

The user will take a single individual SIM card testing device 10, place a physiological sample onto a sample region 14 of the device 10 directed by printed indicia on the surface 12 of the SIM card 10. The system of the present invention provides a protective cover 22 constructed and arranged to hold the sample in place on the SIM card 10. The cover 22 provides both an assurance that the sample region is not further contaminated with other unwanted substances and protects the sample from contaminating the transmission device 40 into which the SIM card 10 is inserted. Once the sample is on a covered SIM card 10 the SIM card 10 is inserted into a transmission device 40. Transmission device 40 is any structure constructed and arranged to receive the SIM card 10, interpret the data from analysis of the sample and transmit that data. In one non-limiting embodiment, as shown in FIG. 4, the transmission device 40 is a cell phone or smart phone.

Transmission can by any acceptable means, including but not limited to telephone transmission, SMS transmission, MMS transmission, USSD, combinations thereof and the like. The actual transmitter is any acceptable device for transmission of data according to the present invention. The transmitter 40 can be any of a stand-alone transmitter, a transmitter/receiver, a cell phone, smart phone, tablet computer and the like. In a preferred embodiment the transmission device is a Global System for Mobile Communications (GSM) phone device. The system of the present invention further provides that transmission is to a receiver 50.

As used herein, “Receiver,” includes any machine configured to receive the information transmitted. Receiver includes, but would not be limited to, a computer database constructed and arranged to receive, organize and present received data to a medical provider. Medical provider, as used herein, refers to any one or combination of, a physician, physician assistant, nurse, nurse practitioner, laboratory, insurance company, combinations thereof, and the like. It is further contemplated in the system of the present invention that the receiver can assemble a historical log of transmissions such that medical tests that are repeated can be presented in chronological order in order to monitor the measured levels of chosen medical tests.

In one embodiment, a log having one or more test results is stored in a database 60 on a non-transitory computer readable medium and database 60 is configured to selectively provide an output 70. Although FIG. 4 demonstrates one output being a report to a physician 70, output is not intended to be limited and includes any output to any person or entity. As contemplated in the present invention, the output is selectively configurable and can further include alerts when threshold levels are breached. As one, non-limiting example, a person utilizing the present invention to test for elevated blood alcohol levels can receive an alert from the system if a test sample is above a particular level. In one embodiment, output is restricted to authorized users.

In another embodiment, output removes identifying information and provides an output of test results not identifiable by the system.

In one embodiment, transmitted data is encrypted prior to transmission and receiver 50 is configured to receive and unencrypt the data.

In one embodiment of the present invention the testing strip is any size, shape, or configuration and is paired appropriately with a transmitter according to the principle of the present invention.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A system for measuring and transmitting measuring a characteristic of a physiological fluid and transmitting said characteristic to a receiver, said system comprising:
a card constructed and arranged to perform analysis of a physiological fluid and said card configured to convert said analysis to an electronic file;
a transmission device constructed and arranged to receive said card and transmit said electronic data file; and
a receiver configured to be operatively associated with said transmission device for receiving said data transmitted thereto, wherein said receiver prepares an output based on reception of said electronic data.

2. The system of claim 1 wherein said transmitter is a cellphone, a smart phone, a tablet computer, or a stand-alone transmitter.

3. The system of claim 1 wherein said electronic data associated with said chemical test is encrypted prior to transmission.

4. The system of claim 1 wherein said physiological fluid is blood, saliva, urine or mucosal fluid.

5. The system of claim 1 wherein said chemical test of said physiological fluid is a qualitative test.

6. The system of claim 1 wherein said chemical test of said physiological fluid is a quantitative test.

7. The system of claim 1 wherein said receiver is operatively associated with said transmitter such that said receiver receives and processes said data transmission.

8. The system of claim 1 wherein said receiver compiling said output configures said output for review.

9. A method for testing biological fluids, formatting test results for transmission, transmitting test data, said method comprising the steps of:
providing a SIM card according to claim 1;
placing a test fluid on a test region indicated on said SIM card;
covering said SIM card with a cover on top of said test fluid;
inserting said SIM card in a device configured for reading test results from said SIM card;
formatting said test results into data for transmission;
transmitting said test results data on a transmitting device for transmitting said data;
receiving said transmitted data on a receiving device configured for receiving said transmitted data;
storing said data on a storage device configured for storing said data.

10. The method of claim 9 wherein said test fluid is at least one of blood, saliva, urine or mucosal fluid.

11. The method of claim 9 further comprising an output of said stored data.
